(19) United States
(12) Patent Application Publication
(10) Pub. No.: US 2003/0039697 A1
(43) Pub. Date: Feb. 27, 2003

(54) Matrices containing nitric oxide donors and reducing agents and their use

(76) Inventors: Yi-Ju Zhao, Ellicot City, MD (US); James A Braatz, Beltsville, MD (US); Gerald M Rosen, Lutherville, MD (US)

Correspondence Address: VENABLE, BAETJER, HOWARD AND CIVILETTI, LLP, P.O. BOX 34385, WASHINGTON, DC, 20043-9998, US

(21) Appl. No.: 10/221,388

(22) PCT Filed: Mar. 20, 2001
(86) PCT No.: PCT/US01/08806

(51) Int. Cl. A61K 38/06; A61K 9/14; A61K 31/21; A61K 33/00

(52) U.S. Cl. 424/486; 424/608; 514/18; 514/509

(57) ABSTRACT

A composition comprises a hydrophobic matrix, a reducible nitric oxide (NO) donor, and an intrinsic reductant reactably associated together with the reducible NO donor within the matrix, and releases an effective amount of NO from the matrix when wetted at physiological pH, independently of the presence or absence of extrinsic reducing agents. The composition inhibits the growth of target cells in a target medium.

### BACKGROUND OF THE INVENTION

This invention relates to matrices that release nitric oxide. In particular, the invention relates to matrices containing a compound that releases nitric oxide (NO) and, optionally, a reducing agent that promotes NO release from the matrix. The invention also relates to uses of such matrices.

There is a widespread need for techniques that improve surface properties of blood-contacting surfaces, e.g. to prevent platelet aggregation and neutrophil adhesion, and to prevent infection, which can result in deleterious effects. By modifying blood-contact properties of such surfaces, one can reduce or eliminate the need for systemic anti-coagulation therapy, extend the life expectancy of long-term implanted blood-contacting devices such as vascular grafts, and improve the performance of shorter-term interventional devices, such as urinary and vascular catheters.

Invasive therapy such as vascular catheterization can be complicated by local infection and induced sepsis, which usually causes the failure of the therapy and is often life-threatening. About 6%˜10% catheters used for long-term venous access become infected (Bernard R W, et al., “Subclavian vein catheterization: a prospective study. II. Infectious complications,” Ann Surg 173:191, 1971; Uldall P R, Joy C, Merchant N., “Further experience with a double-lumen subclavian cannula for hemodialysis, Trans Am Soc Artif Intern Organs 28:71, 1982).

The catheter can allow microorganisms to gain access directly into the patient's vascular system. Biomaterials may alter host humoral and cellular immune response. The relatively hydrophobic property of the biomaterial makes it easy for bacteria to adhere to its surface. Endoscopic catheters and instruments suffer similar problems. Efforts have been made to reduce catheter infection, such as modifying the biomaterial surface to diminish bacterial adhesion, and binding antibiotics to the surface of biomaterials. However, none of these has been successfully used in clinical practice, and administering antibiotics systemically is unsatisfactory. Catheter-induced infection still remains a problem to be solved.

As early as 1927, Warburg's study suggested that nitric oxide could reversibly and irreversibly inhibit the respiratory enzyme of yeast cells, that reversible inhibition could restrain bacteria growth, and that irreversible inhibition may kill bacteria. (Warburg, O., 1927, “The inhibition of carbon monoxide and of nitric oxide on respiration and fermentation,” Biochem Z. 189:354-380). There has been little progress on this front.

Another common complication from the use of inserted devices or devices used for extracorporeal flow of bodily fluids is platelet aggregation and thrombogenesis. There are several known techniques which have been dried to reduce thrombogenicity of medical devices by surface modification or coating. Several types of heparin coatings (covalent and ionic) have been produced. The performance of these coatings has been disappointing and none have been accepted for routine clinical practice. Phosphorylcholine coatings, marketed by Biocompatibles, Ltd., and described in U.S. Pat. No. 5,658,561, are at a very early stage of development and have not been well demonstrated.

Another technique to prevent thrombogenesis is release of NO from polymer films containing nitroso-containing compounds. Espadas-Torre, C., et al., “Thromboresistant chemical sensors using combined nitric oxide release/ion sensing polymeric films,” J. Am. Chem. Soc., 1997, 119:2321-2322. Nitric oxide-containing compounds may be characterized into several groups. (1) N-nitroso compounds are stable and do not readily release NO absent hydrolysis. In addition, N-nitroso compounds present risks of carcinogenicity. (2) A variety of S-nitrosothiols are known to generate NO in vivo. (3) C-nitroso compounds tend to be stable and release NO at body temperature, as in Rosen et al., U.S. Pat. No. 5,665,077. (4) Nitrosyl-containing organometallic compounds are described in Rosen et al., U.S. Pat. No. 5,797,887. According to the latter patent, decomposition of a nitrosyl-containing organometallic compound, such as nitroprusside, into NO is restricted by a polymer coating with a small porosity that inhibits the diffusion of blood-borne reductants to the NO-releasing compound; yet this small porosity allows NO to diffuse through the polymer into the surrounding fluid. There is a need for matrices demonstrating enhanced release of NO.

Green, U.S. Pat. No. 5,944,444, describes release of NO from biodegradable polymer matrices containing nitrites in an acid environment. The picomolar concentrations of NO released are undesirably low, and are not sustained over time. The requirement of a low pH environment is inconsistent with use at physiological pH as in blood and other tissues.

Green et al., U.S. Pat. No. 5,814,666, describes N-nitroso compounds (NONOates) that release NO with antimicrobial effect upon hydrolysis when injected or ingested. Use of NONOates is incompatible with generating NO by reduction.

Polymer matrices containing porosigens taught in the prior art, e.g., Eury, et al., U.S. Pat. No. 5,605,696, designed to facilitate the release of the therapeutic drug from the polymer coating into the vasculature, are unsatisfactory for enhancing nitric oxide release from nitric oxide donors.

Nitroprusside (as in, for example, sodium nitroprusside or SNP) has drawbacks when administered systematically as a NO donor, including short biological half time and systemic effects. There is a need for techniques that would prolong SNP biological effects and limit SNP effects to a local area.

Folts et al., WO 95/07691, describes using S-nitroso and other NO adducts mixed with bovine serum albumin on blood-contacting surfaces to inhibit platelet deposition. Such compositions are not biostable and allow the NO adduct to leach into the blood.

### SUMMARY OF THE INVENTION

The invention relates to compositions that release NO and uses thereof. The self contained system of the invention may be used as a drug delivery device or a coating on a medical device that contacts blood or other body fluids to bring about biological effects. Desired biological effects include preventing aggregation of platelets and inhibiting proliferation of tissue within or near the device (which could decrease functioning of the device), and antimicrobial effects. Further, the favorable effects of the NO release include reducing damage caused by the device itself, and providing a broadened therapeutic benefit.

The invention provides a composition comprising a biostable preferably hydrophobic matrix, a reducible NO donor, and an intrinsic reductant reactably associated together with the reducible NO donor within the matrix, that may release an effective amount of NO from the matrix when wetted in a target medium for a sustained period, independently of the presence or absence of extrinsic reducing agents, and inhibits the release of the NO donor. This invention does not require an acidic pH to release NO from the donor, as is the case in Green, U.S. Pat. No. 5,944,444. The target liquid is preferably at physiological pH. Preferred target media include biological fluids, particularly blood.

The matrix may comprise a polymer. Nitric oxide donors may be nitrosyl-containing organometallic compounds, or S-nitroso compounds. Preferably, the NO donor is a reducible NO donor such as sodium nitroprusside or S-nitrosoglutathione and may be present in an amount between about 0.1% and about 50% and preferably from about 1% to about 10%. Reductants that may be suitable for use in the composition of the invention include ascorbic acid, cysteine, glutathione, penicillamine, N-acetylcysteine, iodine, hydroquinone, mercaptosuccinic acid, thiosalicylic acid, methylthiosalicylic acid, dithiothreitol, dithioerythritol, 2-mercaptoethanol, and FeCl₂. Other reductants presently known or hereafter discovered may be used it they are compatible with the NO donor. The reductant is preferably present in a concentration from about 0.1% to about 25% and preferably between about 1% and about 10%.

In another aspect, the invention is a medical device having as a blood-contacting surface a composition comprising a hydrophobic matrix, a reducible NO donor, and an intrinsic reductant reactably associated together with the reducible NO donor within the matrix, that may release an effective amount of NO from the matrix when wetted in a target medium for a sustained period, independently of the presence or absence of extrinsic reducing agents. The composition of the blood contacting surface of the medical device may be one in which the NO donor is nitroprusside or S-nitrosoglutathione, the matrix comprises silicone, and the reductant is ascorbic acid, cysteine, glutathione, penicillamine, N-acetylcysteine, glutathione, mercaptosuccinic acid, thiosalicylic acid, methylthiosalicylic acid, dithiothreitol, dithioerythritol, 2-mercaptoethanol or FeCl₂.

In yet another aspect, the invention is a composition comprising means for releasing NO in the presence of a reducing agent, and means for incorporating the NO releasing compound with a reducing agent together in a hydrophobic matrix.

In an additional aspect, the invention is a method for improving the performance of a device in a target medium by providing the device with a surface comprising a hydrophobic matrix comprising a compound that releases NO in the presence of a reductant, and associated therewith a reductant, the matrix being capable of releasing NO into the target medium in an amount effective to produce a desired effect. The desired effect may be to: inhibit cell proliferation, retard growth of cancer cells, act as a second messenger in stimulating host immune response toward bacteria, viruses, fungi, parasites and other microbes and cancer cells, promote gastrointestinal motility, stimulate penile erection, relax the uterus during pregnancy, dilate blood vessels, inhibit platelet adhesion, aggregation, and activation, inhibit neutrophil adhesion, and regulate smooth muscle tone. Inhibition of target cell growth is particularly preferred.

In still a further aspect, the invention is a method comprising providing a first compound that releases NO when reduced, providing a second compound that reduces the first compound, the first and second compounds being associated together within a hydrophobic matrix, contacting the hydrophobic matrix with a target medium, allowing the second compound to reduce the first compound so as to produce NO, and selectively allowing the NO to be released from the matrix into the target medium.

In still another aspect, the solid matrix is at the surface of a device, and the step of providing the solid matrix may comprise coating the surface of a device with the solid matrix. The contacting step may comprise inserting the solid matrix into the target medium, or if the solid matrix coats an internal surface of a container such as a vessel or tubing, the contacting step preferably comprises placing the biological medium into the container. The matrix may optionally be withdrawn from the biological medium. The device may be an interventional medical device such as a urinary tract catheter or blood catheter.

The method is effective where the biological medium has a non-acid pH, such that NO is released at a non-acid pH, or a physiological pH (typically neutral or above, although lower in some tissues). Nitric oxide production from the NO donor is not pH dependent.

The solid hydrophobic matrix preferably consists essentially of a matrix forming solid and the nitric oxide donor, or the matrix may comprise a reductant reactably associated with the nitric oxide donor. The solid matrix is preferably formed by a hydrophobic polymer, which may be one or more selected from the group consisting of silicone, polyvinylchloride, polystyrene, PMMA, polyolefins, and polytetrafluorocarbons. In one embodiment, toxic byproducts are produced with the nitric oxide from the nitric oxide donor and the solid matrix inhibits release of the toxic byproducts.

The nitric oxide donor is preferably nitroprusside. The NO donor may be S-nitrosoglutathione. One or more donors may be used depending on the circumstances.

A biological medium is a preferred target medium. The biological medium is preferably a biological fluid such as blood or urine or interestitial fluid. It may be a non-fluid tissue such as skin, cells, or a urethral lining.

The target cells are preferably one or more selected from the group consisting of bacteria, fungi, virally infected cells, parasitic microorganisms, and cancer cells. The method is preferably effective such that the growth rate inhibition is at least about 25%, preferably about 50%, or greater than about 90%. In most preferred embodiments, the method kills target cells. More particularly, the method may extend the length of time for 50% of saturation to occur (T₅₀) in a growth medium by 25%, 50%, double, or longer. The method may reduce the count of cells that grow on a surface such as an interventional catheter within a given period by 25%, 50%, or 90%. Most preferably, the method completely prevents growth of cells on such surfaces.

In other aspects of the invention, a method comprises: providing a device coated with a solid hydrophobic matrix comprising a NO donor retained within the matrix; and contacting the coated device with a target medium containing target cells; the NO donor reacting within the matrix to produce NO, and the NO, but not the NO donor, being released from the solid hydrophobic matrix and thereby inhibiting growth of target cells in the vicinity of the device.

A method of using a medical device in a biological medium according to the invention comprises: a step for achieving contact between the medical device and the target medium; a step for producing NO non-hydrolytically from a NO donor within a solid matrix at the surface of the device; and a step for releasing NO from the device in the target medium over a sustained period without releasing the NO donor, in an amount effective to inhibit growth of target cells in the target medium.

According to an embodiment of the invention, a target medium contacting surface is provided that releases NO, thereby having improved properties such as that it is less susceptible to thrombosis and infection, and thus has reduced occlusion and lower likelihood of failure. Compositions that include Nitrosyl-containing organometallic compounds or S-nitrosothiols that release NO upon reaction with a reductant may be reactably associated with a reductant in a matrix, preferably a hydrophobic polymer, present as a coating or at a device surface. In these systems, the nitrosyl-containing organometallic compound is preferably nitroprusside, the S-nitrosothiol is preferably S-nitrosoglutathione, the hydrophobic polymer matrix preferably comprises silicone, and the reductant is preferably ascorbic acid or glutathione. The coating inhibits the diffusion into the polymer matrix of blood-borne reductants, but is nonetheless able to release NO without exposure to light or hydrolysis.

A further embodiment of the invention envisions providing a tissue contacting surface that releases NO, thereby having improved properties such that it is less susceptible to infection, and has lower likelihood of failure by for example, inhibiting cell proliferation such as myointimal hyperplasia. Such a coating is able to release NO without hydrolysis. Nitric oxide may be generated by reduction, thermolysis, nucleophilic decomposition, electrophilic decomposition, catalysis and combinations thereof. Reduction is a preferred pathway for generating nitric oxide; thus, preferred nitric oxide releasing compositions include a reductant.

The claimed invention relies on a specific kind of NO donor: a therapeutic agent precursor that produces NO in therapeutic amounts, such as SNP or S-nitrosoglutathione (GSNO). Preferred compositions include a reductant such as ascorbate, retained together with the NO donor in the matrix. Decomposition of SNP or GSNO by ascorbic acid within the matrix produces a by-product, NO. It is NO, not SNP or GSNO, which diffuses from within the polymer into the blood stream or other bodily fluids.

Advantages of this invention include:

1) Toxic byproducts of NO donor decomposition, such as cyanide in the case of nitroprusside, may be trapped in the coating, preventing or reducing toxic response to these byproducts.

2) Release of effective amounts of NO according to the invention occurs within a controlled solid matrix, and does not involve releasing the NO donor into the biological medium to generate NO there, under poorly controllable conditions.

Additional advantages of compositions according to the invention that contain a reducing agent in the matrix include:

3) NO release does not depend on exterior reducing agents, light or hydrolysis. It can provide a controlled release of NO by varying the concentration of the reductant in the polymer that is applied onto the surface of implanted devices and catheters.

4) The inventive methods of using coatings and devices permit more accurate design and control of NO release than was previously possible. The release is independent of the individual patient's metabolic conditions. There is preferably no need for light, hydrolysis or additional coating components to bring about NO release.

The invention differs from the prior art in the use of nitrosyl-containing organometallic compounds, S-nitroso compounds, and C-nitroso compounds as nitric oxide-releasing antimicrobial agents, in a device coating with a biostable matrix that includes and retains such compounds, where the device exhibits cytotoxic or cytostatic effects.

This invention provides advantages that were not previously appreciated, including the possibility of exactly controlling the NO release pattern without regard to individual patient blood characteristics or hydrolytic pathways for generating NO and the possibility of reducing systemic use of antibiotics in conjunction with invasive medical procedures.

This invention satisfies a long felt-need for insertable medical devices that do not promote infection, and can instead reduce microbial growth and promote other desirable properties. This invention is contrary to the teachings of the prior art such as Green, U.S. Pat. No. 5,814,666 which disfavored nitrosyl-containing organometallic compounds such as sodium nitroprusside because they require activation to release NO.

In some compositional aspects, this invention differs from the prior art in modifications that were not previously known or suggested. The compositions in the prior art lack reductants along with NO donors, release NO donors into the target medium or release NO donors through hydrolytic pathways.

Compositions of the invention satisfy a long felt need for a composition that releases NO in a controlled pattern. This invention is contrary to the teachings of the prior art in that it associates nitroso-containing compounds with reductants in the polymer to release NO, whereas the prior art taught inhibiting the ability of reductants to diffuse into the polymer.

Further objectives and advantages will become apparent from a consideration of the description, drawings, and examples.

### BRIEF DESCRIPTION OF THE DRAWINGS

The invention is better understood by reading the following detailed description with reference to the accompanying figures and tables, in which like reference numerals refer to like elements throughout, and in which

FIG. 1 shows NO release from SNP/Si coating containing 1% L-ascorbic acid (LAA) in the dark.

FIG. 2 shows NO release from SNP/Si coating containing 10% L-ascorbic acid (LAA) in the dark.

FIG. 3 shows NO release from GSNO/Si coating containing 3% L-ascorbic acid (LAA) in the dark.

FIG. 4 shows the inhibitory effects of SNP/Si coating on S. aureus growth.

FIG. 5 shows the inhibitory effects of SNP/Si coating on S. aureus growth starting from a lower bacterial concentration.

FIG. 6 shows the inhibitory effects of SNP/Si coating on E. coli growth.

FIG. 7 shows the inhibitory effects of SNP/Si coating on E. coli growth starting from a lower bacterial concentration.

FIG. 8 shows that SNP and GSNO with a reducing agent inhibits growth of bacteria.

### DETAILED DESCRIPTION OF THE PREFERRED EMBODIMENTS

In describing preferred embodiments of the present invention illustrated in the drawings, specific terminology is employed for the sake of clarity. However, the invention is not intended to be limited to the specific terminology so selected. It is to be understood that each specific element includes all technical equivalents which operate in a similar manner to accomplish a similar purpose. Each reference cited here is incorporated by reference as if each were individually incorporated by reference.

A device according to the invention may be a medical, veterinary, or laboratory device having a surface that contacts a biological medium in use. These include blood vessel and urinary tract implants such as catheters, stents, intracorporeal or extracorporeal blood circuits, endoscopy equipment, insertable laparascopic devices, implants of bone, polymer, metal, or composites, artificial joints, membranes, tubing, grafts, and other devices inserted into biological media. The materials from which these devices may he made include plastic, stainless steel, nitinol, dacron, polytetrafluoroethylene, and countless other materials known to practitioners.

A “NO donor” refers to a compound that releases NO on decomposition. A “reducible NO donor” refers to a nitrosyl-containing compound that releases NO in the presence of a reducing agent under the mild conditions encountered within a biostable hydrophobic polymer matrix. In general, NO donors include reducible NO donors and others.

A target cell is any cell or cell population that is targeted for growth inhibition or killing. Examples include bacteria, fungi, viruses, parasitic microorganisms, cancer cells, and cells that are foreign or undesirable in a patient animal such as a human or animal. Growth inhibition means that the method results in a growth rate slower than that which would be present in the absence of the inventive method. The extent of inhibition may be small or complete, and the method may involve killing cells (reversing the growth of the population).

The target medium is one that does not prevent the NO donor from reacting within the matrix to produce NO and release it into the medium. Nitric oxide is generally considered hydrophobic. Typically the target medium is a biological medium, such as an aqueous liquid like blood, urine, interstitial fluid, or cell growth medium in vitro. The liquid is preferably at physiologic pH or is pH neutral, i.e. having a pH greater than about 5, and most preferably has a pH of about 7 or slightly above, such as blood. The medium may also be tissue such as skin, internal tracts, or interstitial tissue.

Nitrosyl-containing organometallic compounds, such as sodium nitroprusside, are readily susceptible to reduction, and are preferred. S-nitroso compounds, such as S-nitrosoglutathione, may be paired with a suitable reducing agent in a matrix according to the invention, and are preferred as well. Preferably, the release of NO from the NO donor is not pH dependent. The practitioner will be able to use such nitrosyl-containing organometallic or S-nitroso compounds, selecting those that generate NO in the presence of a reducing agent and a hydrophobic matrix, without toxic byproducts.

The reaction which generates NO from a NO donor is preferably non-hydrolytic because there is no water present or limited amounts present in the solid phase of the biostable matrix. For reducible NO donors, NO is generated and released in effective amounts by reduction, although other mechanisms may also operate to a limited extent, such as photolysis, thermolysis, hydrolyis, or other mechanisms. This is in contrast to use of nitrites and NONOates, and other compounds that generate NO primarily by hydrolysis. Reductive degradation of reducible NO donors in the presence of reductants according to the invention does not preclude generating NO to some extent by other mechanisms.

Reducing agents according to the invention include ascorbic acid and others that are effective to reduce the reducible NO donor in the polymer matrix. The reductant must be selected to be compatible with the reducible NO donor. Examples of other reducing agents include cysteine, penicillamine, N-acetylcysteine, glutathione, mercaptosuccinic acid, thiosalicylic acid, methylthiosalicylic acid, dithiothreitol, dithioerythritol, 2-mercaptoethanol, and FeCl₂.

A biostable matrix according to the invention is preferably hydrophobic, that is, one that absorbs a limited amount of water, preferably less than 10-20%, although other, less hydrophobic polymers absorbing 50% or 100% of their weight in water, or higher, may also be suitable according to the invention. Any biostable matrix is useable as long as it retains the nitric oxide donor, reductant, if present, and other reactants and by-products, while releasing nitric oxide, and prevents unwanted or uncontrolled reactions resulting from water penetration. The matrix may be hydrated before contacting the biological medium. Polymer matrices are preferred for their simplicity, although ceramic or other types of alloys could accomplish the same function. Silicone is a preferred polymer. Other hydrophobic polymer examples include but are not limited to: PVC, polystyrene, polymethylmethacrylate (PMMA), polyolefins, polyfluorocarbons, etc. When reducible NO donors are used, the hydrophobic matrix must entrap and retain the reducible NO donor and reductant together in a reactive relationship so they are not released in a significant amount, but must permit the NO to be released. For example, a polyurethane matrix releases ascorbic acid and is therefore incompatible with the inventive compositions absent modification according to the invention.

The matrix is biostable in that it is not appreciably biodegradable or bioabsorbable. The matrix inhibits release of the reductant, the NO donor, toxic and other reactants and byproducts during an effective period of use from several minutes to several months, preferably at least about 12 hours, and more preferably at least about one day.

The matrix is biostable, meaning that it does not degrade in the target medium particularly when the target medium is a biological medium. Of course, the stability relates to the medium and some media and uses require a more durable matrix. If the matrix is not sufficiently stable it will either physically wear off or slough off, or dissolve, or degrade chemically in the medium, yielding uncertain dosage and uncontrolled release of NO donor and by-products. The matrix is selected so that it can retain the NO donor and reductant for an effective product life, allow them to react to produce NO, and allow the NO to be released from the matrix. Thus, the invention employs a self-contained solid phase NO releasing system that is not dependent on the nature of the target medium or reactions that may occur in it, to produce desirable biological effects.

The invention permits effective concentrations of NO to be released into a physiological environment over a sustained period. The amount of components released from the matrix into a medium depends on their concentration, the rate of release, and time. It is important that there is no deleterious effect from the release of any component from the matrix, either on the medium itself, or in terms of interfering with desirable effects of NO. The matrix inhibits the release of the NO donor and preferably there is no release of other components such as the optional reductant, NO donor, or byproducts other than NO that would cause a discernable deleterious effect or interference with the NO.

Preferably, the amount of NO released is greater than about 10 nmoles. Sustained release in this context means that the concentration does not drop below a threshold of effectiveness and/or remains within a certain proportion of the initial concentration for a suitable period. For example, in some applications it is desirable that the concentration not drop by more than one order of magnitude, e.g., 1 nmole, over a two week period. In other applications the period of sustained release may need to be shorter (e.g. minutes) or longer (e.g. months). In yet other applications, the effective range may be broader.

In its compositional aspects, the invention provides a new NO releasing mechanism. The NO donor, preferably nitroprusside or S-nitrosoglutathione, reacts with the intrinsic reducing agents, and generates NO at a more rapid rate than that described in Rosen, U.S. Pat. No. 5,797,877. Nitric oxide is released, and nitroprusside, for instance and reducing agents, as well as the byproducts of nitroprusside decomposition, are trapped in the polymer matrix. This NO releasing mechanism is confirmed by the following experimental results detailed in the examples:

1. Pores created by washing out lactose did not improve NO release from SNP in a silicone coating.

2. A SNP/silicone coating plus L-ascorbic acid (either 1% or 10%) did release NO in the dark.

3. A GSNO/silicone coating plus L-ascorbic acid (LAA, 3%) did release NO in the dark, and release of NO was considerably greater than GSNO in the absence of L-ascorbic acid.

Thus, the reducible NO donors, SNP and GSNO, when incorporated into a silicone coating with reducing agents release NO at a rate greater than SNP or GSNO alone. They are also cytostatic and/or cytotoxic.

The antimicrobial method aspect of the invention is intended not to produce toxicity to healthy cells of the target animal or patient in in vivo applications. The effective amount of NO to be released depends on the target cells, the target medium, and the desired degree of inhibition or killing, and the sensitivity of the host tissue, as can readily be determined by a person of ordinary skill. Specifically excluded from the meaning of inhibition of target cell growth in this context is inhibition of platelet aggregation as known in U.S. Pat. No. 5,797,887, which is not a proliferation cell growth phenomenon. Thus, the inventive method relates to inhibition of non-platelet target cell growth. In this application, inhibition of platelet aggregation and anti-restenosis effects are referred to specifically but not as inhibition of target cell growth.

The invention is better understood upon consideration of the following non-limiting examples illustrating preferred embodiments of the invention. Periods skilled in the art may identify other embodiments which are within the scope of the invention upon consideration of the examples.

### EXAMPLES

Materials

Silicone

RTV-12A 01G, from GE, Batch# HB156

RTV-12C 01P, from GE, Batch# HD213

L-Ascorbic Acid, from Sigma, Lot# 48H1038

L-Cysteine, from Sigma, Lot # 107H09382

Glutathione, from Sigma, Lot# 48H3502

Sodium nitroprusside (SNP), from Sigma, Lot# 96H3502

S-nitroso-L-glutathione, Lot #125H4124

Sulfanilamide, from Sigma, Lot# 77H0150

N-(1-Naphthyl)ethylenediamine, from Aldrich, Lot# 01715LW

24 well untreated tissue culture plate from Becton Dickinson Labware Lot# 17348

Phosphate buffered saline (PBS) from Sigma, Lot 88H6073 (NaCl 120 mM, KCl 2.7 mM, and phosphate buffer 10 mM, pH 7.4 at 25° C.)

Griess Reagents

RT1: dissolve 5 g Sulfanilamide in 500 ml 5% H₃PO₄

RT2: dissolve 0.5 g N-(1-Naphtyl) ethylenediamine in 500 ml distilled water.

Mix RT1 and RT2 in the ratio 1:1 before use.

Methods

Method I Plate Coating

RTV-12A and RTV-12C were mixed in a ratio of 20:1 (v/v) and 0.2 ml of the silicone mixture was added to wells of a 24 well plate. Other additives, such as SNP, GSNO, reducing agents, or lactose were added in different experiments. The coating procedure was done at room temperature and in reduced light.

Method 2 Nitrite Assay

Accumulation of nitrite was determined colorimetrically by mixing 0.5 mL each of culture medium and freshly prepared Griess reagent [0.1% N-(1-naphthyl)ethylenediamine in water and 1% sulfanilamide in 5% phosphoric acid, mixed 1:1] (Green, et al., Anal. Biochem 126, 131-138, 1982.). Concentrations of nitrite were estimated by comparing absorbance at 550 nanometers against standard solutions of sodium nitrite prepared in the same medium. Nitrite indicates presence of nitric oxide and/or nitroprusside.

### EXAMPLE 1

SNP, a NO donor according to the invention, is retained within a solid silicone matrix, even if it is rendered porous by including lactose as a porosigen in the matrix and then washing out the lactose. Lactose (1% and 10%, w/v) was added to SNP and silicone mixtures that were added to wells of a 24 well plate. PBS was added to each of the coated wells. The plate was wrapped with foil and placed in the dark. A sample was collected every 24 hours for nitrite assay, and the buffer was replaced with fresh PBS. No significant nitrite concentrations were detected in the samples over a ten-day test period. The results demonstrate that even with voids left from washed out lactose, a silicone matrix did not release SNP into the medium.

### EXAMPLE 2

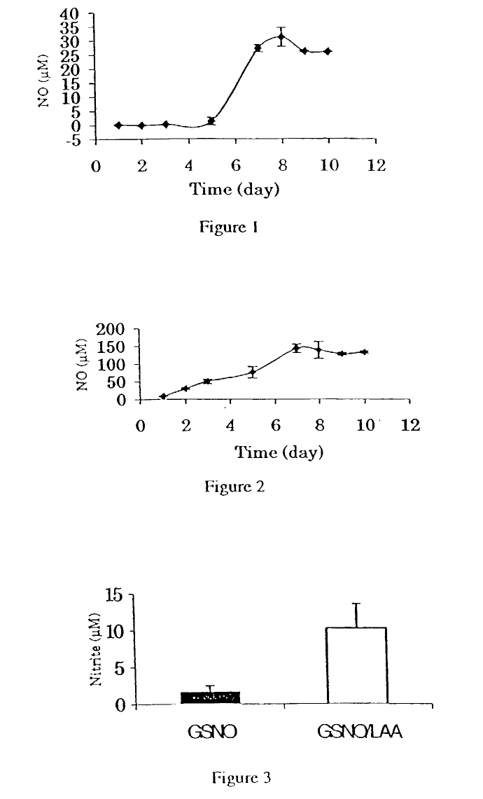

The reducing agent L-ascorbic acid improves NO generation from a hydrophobic matrix containing the NO donor, SNP. L-ascorbic acid was added to a SNP/Si coated surface. In the same experimental conditions as mentioned above, that is, in the dark, SNP/Si plus L-ascorbic acid coatings released NO in a dose-dependent manner (FIGS. 1 and 2). Nitric oxide production reached a peak at 7-8 days with 1% and 10% L-ascorbic acid. Peak concentrations were 32 μM and 150 μM, respectively.

The effectiveness of L-ascorbic acid in increasing NO release is in contrast to the lack of effect of lactose, as shown above. These data suggest that porosigen effects did not contribute to NO produced in SNP/Si plus L-ascorbic acid coatings.

Further, there is evidence to show that SNP/Si plus L-ascorbic acid coatings release NO rather than SNP itself. First, SNP without reductant is not released as shown above.

Second, if SNP itself were being released, a first order decline should be observed day by day as the NO donor concentration in the matrix diminishes. To the contrary, in this experiment, NO release into the fresh buffer increases with time, which is inconsistent with leaching of SNP from the matrix. Rather, there is a second order effect perhaps as NO accumulates in the matrix, although the mechanism is unclear.

### EXAMPLE 3

The reducing agent L-ascorbic acid improves NO generation from a hydrophobic matrix containing the nitric oxide donor, GSNO. L-ascorbic acid was added to a GSNO/Silicone coated surface. In the same experimental conditions as mentioned above, that is, in the dark, GSNO/Silicone produced only 2 μM of NO after 1 day. In contrast, GSNO/Silicone plus L-ascorbic acid coated surface released 10 μM NO after 1 day (FIG. 3).

### EXAMPLES 4-5

Materials and Methods

Tryptic Soy Agar (4% w/v) and Tryptic Soy Broth (30% w/v), Becton Dickinson, containing digested casein, soy powder, and dextrose

VWR Sterile Petri Dish (Polystyrene), 100×15 mm

Flask Coating: Silicones RTV 12A and RTV 12 C were mixed in a ratio 20:1 (v/v). SNP powder was mixed with RTV mixture; 10 ml RTV mixture or 10 ml SNP/RTV mixture was put into each flask and cured 24 hours in dark. All procedures were performed in reduced light and room temperature.

Nitric oxide release from SNP/Si coating: The coated flask was filled with PBS, or TSB 15 ml. The flasks were placed in a shaking incubator, shaking speed 200 RPM @ 37° C. Samples were collected for nitrite assay. A curve of accumulation of nitrite was generated.

Bacterial growth curve: 15 ml TSB was placed in each flask. Equal amount of bacteria was added to each flask. The flasks were placed in a shaking incubator, shaking 200 RPM @ 37° C. Samples were collected for O.D. measurement. An accumulation curve were generated.

Bacterial growth on agar: 4 grams TSA was dissolved in distilled water, and autoclaved at 121° C. for 15 minutes. When the agar cooled to 50° C., 15 ml agar was placed into each tube, and equal amounts of bacteria were added to each. Then the agar and bacteria mixture was cast on culture dishes. The dishes were placed into an incubator @ 37° C. The clone number was counted at 24 hours.

### EXAMPLE 4

SNP/silicone coatings inhibit bacteria growth. Flasks were coated with silicone containing 1%, 5%, and 10% SNP (w/v). A flask coated with only silicone was used as control (see method 1). Light absorbency was measured (@ 600 nm) to evaluate bacteria growth.

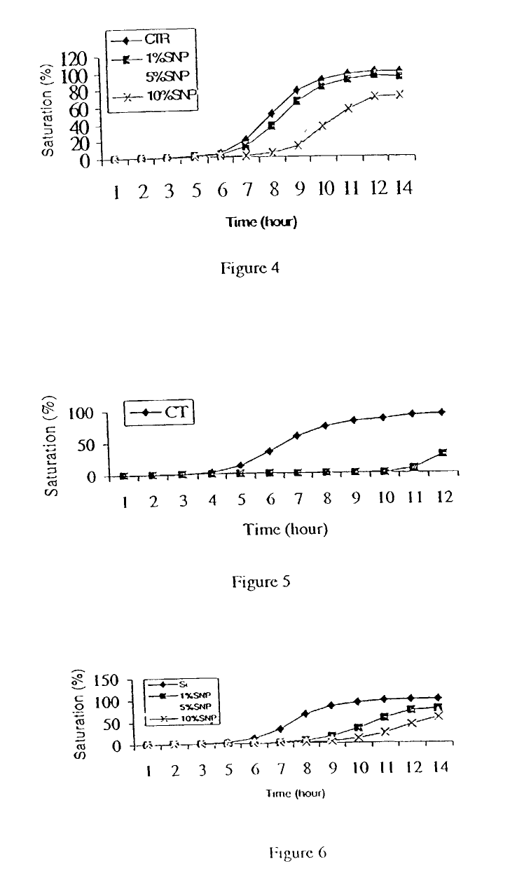

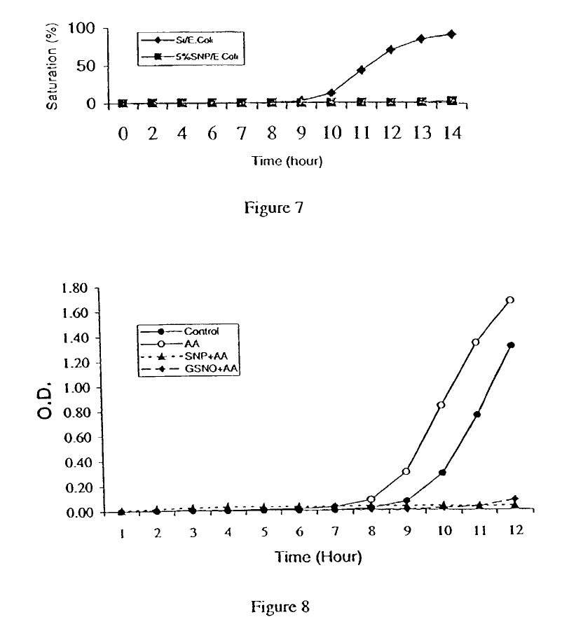

FIGS. 4 and 5 present the results of experiments with S. aureus. FIGS. 6 and 7 show the results of experiments with E. coli. A very high titer of bacteria, about 400,000 cells, was transferred to each flask (FIGS. 4, 6). Compared with control, SNP/Si coating inhibits the growth of S. aureus and E. coli in a dose-dependent manner. At even 100 times higher starting concentration of bacteria, a dose-dependent effect was still noted, but the effect was less dramatic than shown in FIGS. 4 and 6 due to saturation. These experiments were repeated with about 1000 bacteria introduced at the beginning (FIGS. 5, 7). Here, the presence of SNP at 5% produced dramatic inhibition of bacterial growth. These results show that 1) SNP/Si coating inhibits bacteria growth, both S. aureus and E. coli (gram-positive and gram-negative, respectively); 2) the inhibition is SNP concentration-dependent; and 3) the inhibition effect is related to bacteria number—higher concentrations of SNP, and presumably of NO, are needed to inhibit very high bacterial number.

It was noted that there was NO release from SNP/silicone in TSB at SNP concentrations as low as 1%. In contrast, in PBS, NO was released at 10% SNP. This establishes that different concentrations of NO donor may be required to achieve effective concentrations in different biological systems.

### EXAMPLE 5

Nitric oxide release from SNP inhibits bacterial growth on agar. Agar containing different concentrations of SNP was used to test the effects of NO release from SNP on bacteria growth. Both S. aureus and E. coli were tested. After 24 hours culture, bacteria number were counted. No bacteria were found in the dishes containing 5% and 10% SNP. Bacterial numbers in dishes of control and 1% SNP were counted. With both S. aureus and E. coli, the experiment showed that 1% SNP inhibits both strains of bacteria, significantly, and 5% and 10% SNP kill S. aureus and E. coli completely; no bacterial growth was observed. These results support the existence of a dose-dependent relationship between release of NO from a nitrosyl-containing organometallic compound and cell growth inhibition. The results also support the use of matrices that are less hydrophobic than silicone.

### EXAMPLE 6

Segments of polyurethane catheter for extracorporeal blood dialysis (available from Bard Access) were coated by dipping in a solution of silicone in tetrahydrofuran and with or without the other components, and allowed to dry. The dipping process was repeated three times. The coatings tested were: 1) silicone, as control; 2) silicone plus 1% (w/v) L-ascorbic acid (AA) as control; 3) silicone plus 5% (w/v) SNP and 1% AA; and 4) silicone plus 1% S-nitrosoglutathione (GSNO) (Sigma) and 1% AA. The coated catheter segments were placed in 15 ml plastic test tubes containing 10 ml Tryptic Soy Broth. An equal amount of E. coli was added to each tube. The tubes were put in a shaking incubator. The speed was set at 200 RPM, and temperature 37° C. Samples were collected for O.D. measurement every hour. Cumulative growth curves were plotted.

The experimental results are shown in FIG. 4. The controls (silicone and ascorbic acid) showed classical growth over a 12-hour period. In contrast, the test samples were effective in eliminating growth of bacteria during the time period of the study. Similar results would be expected for S. aureus and other microbes. Also, the enhanced release of NO from the coated catheter surfaces would have other desirable biological effects such as preventing platelet aggregation.

The embodiments illustrated and discussed in this specification are intended only to teach those skilled in the art the best way known to the inventors to make and use the invention. Nothing in this specification should be considered as limiting the scope of the present invention. The above-described embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

What is claimed is:

1. A composition comprising:
a biostable matrix,
a reducible nitric oxide donor, and
an intrinsic reductant reactably associated together with the reducible nitric oxide donor within the matrix, the nitric oxide donor and reductant generating nitric oxide in a target medium, and the matrix releasing an effective amount of nitric oxide into the target medium, and inhibiting release of the nitric oxide donor into the target medium.

2. The composition of claim 1, wherein the matrix comprises a polymer.

3. The composition of claim 1, wherein the reducible nitric oxide donor is a nitrosyl-containing organometallic compound.

4. The composition of claim 1, wherein the reducible nitric oxide donor is nitroprusside.

5. The composition of claim 1, wherein the reducible nitric oxide donor is S-nitrosothiol.

6. The composition of claim 1, wherein the reducible nitric oxide donor is S-nitrosoglutathione.

7. The composition of claim 1, wherein the reductant is selected from the group consisting of ascorbic acid, cysteine, glutathione, penicillamine, N-acetylcysteine, iodide, hydroquinone, mercaptosuccinic acid, thiosalicylic acid, methylthiosalicylic acid, dithiothreitol, dithiocrythritol, 2-mercaptoethanol, and FeCl₂.

8. The composition of claim 1, wherein the reductant concentration is between about 0.1% and about 25%.

9. The composition of claim 1, wherein the reductant concentration is between about 1% and about 10%.

10. The composition of claim 1, wherein the reducible nitric oxide donor concentration is between 0.1% and about 50%.

11. The composition of claim 1, wherein the reducible nitric oxide donor concentration is between about 1% and about 10%

12. The composition of claim 1, wherein the matrix is hydrophobic.

13. The composition of claim 2, wherein the polymer comprises silicone.

14. A device having a surface comprising the composition of claim 1.

15. The device according to claim 14, wherein the matrix is coated on the device.

16. A medical device according to claim 14, wherein the nitric oxide donor is nitroprusside, the matrix comprises silicone, and the reductant is selected from the group consisting of ascorbic acid, cysteine, glutathione, penicillamine, N-acetylcysteine, glutathione, mercaptosuccinic acid, thiosalicylic acid, methylthiosalicylic acid, dithiothreitol, dithioerythritol, 2-mercaptoethanol, and FeCl₂.

17. The composition of claim 1, wherein the reaction between the nitric oxide donor and the reductant produces toxic byproducts and the matrix inhibits release of the toxic byproducts.

18. The composition of claim 1, wherein the target medium is an aqueous fluid.

19. The composition of claim 1, wherein the target medium is a biological fluid.

20. The composition of claim 1, wherein the target medium is a biological nonfluid.

21. The composition of claim 1, wherein the nitric oxide is released at physiological pH.

22. A composition comprising:
means for generating nitric oxide in the presence of a reducing agent,
means for reducing the nitric oxide releasing means,
means for associating the nitric oxide generating means and the reducing means reactably together in a solid phase such that they interact to generate effective amounts of nitric oxide over a sustained period,
means for releasing the nitric oxide from the associating means, and
means for retaining the nitric oxide generating means within the associating means.

23. A method for improving the performance of a device in a target medium comprising: providing the device with a surface comprising a biostable matrix comprising a compound that releases nitric oxide in the presence of a reductant, and associated therewith a reductant, the matrix being capable of releasing nitric oxide into the medium in an amount effective to produce a desired effect.

24. The method according to claim 23, wherein the desired effect is one or more effect selected from the group consisting of inhibiting cell proliferation, retarding growth of cancer cells, acting as a second messenger in stimulating host immune response toward bacteria, viruses, fungi, parasites and other microbes and cancer cells, killing or inhibiting the growth of bacteria, viruses, fungi, parasites and other microbes and cancer cells, promoting gastrointestinal motility, stimulating penile erection, relaxing the uterus during pregnancy, dilating blood vessels, inhibiting platelet adhesion, aggregation, and activation, and inhibiting neutrophil adhesion and regulating cardiac contractility.

25. The method according to claim 23, wherein the desired effect is inhibiting the growth of target cells.

26. A method of producing a therapeutic nitric oxide effect in a patient in need thereof comprising the steps of:
providing a solid composition matrix comprising a reducible oxide donor and an intrinsic reductant reactably associated together with the reducible nitric oxide donor in a biostable hydrophobic matrix; and
inserting the solid matrix into the patient wherein nitric oxide is released after inserting.

27. The method according to claim 26, wherein the nitric oxide effect is selected from the group consisting of inhibiting cell proliferation, retarding growth of cancer cells, acting as a second messenger in stimulating host immune response toward, or directly inhibiting growth of, bacteria, viruses, fungi, parasites and other microbes and cancer cells, promoting gastrointestinal motility, stimulating penile erection, relaxing the uterus during pregnancy, dilating blood vessels, inhibiting platelet adhesion, aggregation, and activation, and inhibiting neutrophil adhesion and regulating cardiac contractility.

28. A method comprising:
providing a first compound that releases nitric oxide when reduced,
providing a second compound that reduces the first compound, the first and second compounds being associated together within a hydrophobic matrix,
contacting the hydrophobic matrix with a target medium, and
allowing the second compound to reduce the first compound so as to produce nitric oxide, and selectively allowing the nitric oxide to be released from the matrix into the liquid medium.

29. A method of inhibiting the growth of target cells in a target medium, comprising the steps of: providing a solid, biostable, matrix comprising a reducible nitric oxide donor and intrinsic reductant retained within the matrix;
contacting the solid matrix with the target medium;
thereafter, the reducible nitric oxide donor and reductant generating nitric oxide in the target medium, the nitric oxide donor being retained within the matrix, and the nitric oxide being released from the solid matrix in an amount effective to inhibit growth of the target cells.

30. The method of claim 29, wherein the nitric oxide donor is a nitrosyl-containing organometallic compound.

31. The method of claim 29, wherein the nitrosyl-containing organometallic compound is nitroprusside.

32. The method of claim 29, wherein the reductant has a concentration in the range of about 1% to about 10%.

33. The method of claim 29, wherein the nitric oxide donor is a S-nitrosothiol.

34. The method of claim 33, wherein the S-nitrosothiol is S-nitrosoglutathione.

35. The method of claim 29, wherein the step of providing the solid matrix comprises coating the surface of a device with the solid matrix.

36. The method of claim 29, wherein the contacting step comprises inserting the solid matrix into the target medium.

37. The method of claim 29, wherein the device is an interventional medical device.

38. The method of claim 29, wherein nitric oxide production from the nitric oxide donor is not pH dependent.

39. The method of claim 29, wherein the solid matrix is a hydrophobic polymer.

40. The method of claim 29, wherein the hydrophobic polymer is selected from the group consisting of silicone, polyvinylchloride, polystyrene, PMMA, polyolefins, and polytetrafluorocarbons.

41. The method of claim 29, wherein byproducts are produced with the nitric oxide and the solid matrix inhibits release of the byproducts.

42. The method of claim 29, wherein the target medium is selected from a biological fluid and non-fluid tissue.

43. The method of claim 29, wherein the growth rate inhibition is at least about 50%.

44. The method of claim 29, wherein target cells are killed.

45. A method of inhibiting the growth of target cells comprising:
providing a device coated with a solid biostable matrix comprising a nitric oxide donor retained within the matrix;
contacting the coated device with a target medium containing target cells;
the nitric oxide donor reacting non-hydrolytically within the matrix to produce nitric oxide, and the nitric oxide, but not the nitric oxide donor, being released from the matrix and thereby inhibiting growth of target cells.

46. The method of claim 45, wherein the target medium is blood, urine, interstitial fluid, or other biological fluid.

47. The method of claim 45, wherein the target cells are one or more selected from the group consisting of bacteria, fungi, virally infected cells, parasitic microorganisms, and cancer cells.

48. The method of claim 45, wherein the nitric oxide donor is a nitrosyl-containing organometallic compound.

49. The method of claim 45, wherein the nitric oxide is released at physiological pH.

50. A method of using a medical device in a biological medium comprising:
step for achieving contact between the medical device and the biological medium;
step for producing nitric oxide non-hydrolytically from a nitric oxide donor within a solid matrix at the surface of the device; and
step for releasing nitric oxide from the device in the biological medium over a sustained period without releasing the nitric oxide donor, in an amount effective to inhibit growth of target cells in the biological medium.
